(19) United States
(12) Patent Application Publication
(10) Pub. No.: US 2025/0345251 A1
(43) Pub. Date: Nov. 13, 2025

(54) IMPROVEMENTS IN OR RELATING TO ORGANIC COMPOUNDS

(71) Applicant: GIVAUDAN SA, VERNIER (CH)

(72) Inventor: Emmanuel AUSSANT, Argenteuil (FR)

(21) Appl. No.: 18/869,152

(22) PCT Filed: Jun. 20, 2023
(86) PCT No.: PCT/EP2023/066538

§ 371 Date: Nov. 25, 2024

(30) Foreign Application Priority Data
Jul. 14, 2022 (GB) 2210341.0

(51) Int. Cl. A61K 8/11; A61Q 13/00

(52) U.S. Cl. CPC A61K 8/11; A61Q 13/00

(57) ABSTRACT

The present invention provides a microcapsule composition in the form of a slurry, comprising a plurality of core-shell microcapsules comprising a core comprising at least one functional material and a shell encapsulating the core, wherein the core-shell microcapsules represent about 25 wt % to about 50 wt % of the composition; and an aqueous phase, wherein the aqueous phase comprises a monovalent and/or a divalent inorganic salt, and wherein the conductivity of the microcapsule composition is above about 5000 μS/cm.

The present invention relates to microcapsule compositions in the form of a slurry comprising a plurality of core-shell microcapsules and an aqueous phase. In particular, the invention relates to microcapsule compositions comprising a plurality of core-shell microcapsules comprising a core comprising at least one functional material and a shell encapsulating the core; and an aqueous phase, wherein the aqueous phase comprises a monovalent and/or a divalent inorganic salt.

### BACKGROUND OF THE INVENTION

It is known to incorporate encapsulated functional materials in consumer products, such as household care, personal care and fabric care products. Functional materials include for example fragrances, cosmetic actives, and biologically active ingredients, such as biocides and drugs.

Microcapsules that are particularly suitable for delivery of such functional materials are core-shell microcapsules, wherein the core comprises the functional material and the shell is impervious or partially impervious to the functional material. Usually, these microcapsules are used in aqueous media and the encapsulated functional materials are hydrophobic. It is desirable that the shell material has no reactivity with the functional material, is inexpensive, and shows consistent properties during storage.

A broad selection of materials such as aminoplast resins, polyurea resins, polyurethane resins, polyacrylate resin, and combinations thereof have been employed for encapsulating functional materials, especially volatile functional materials, such as fragrance ingredients. Encapsulated perfume compositions are typically prepared in the form of aqueous slurries.

It is important to ensure that the perfume-containing microcapsules are well dispersed in the slurry, and it is particularly important to avoid agglomeration of the microcapsules in the aqueous dispersing medium, in order to prevent flocculation, coagulation, creaming or sedimentation, which may pose an issue for the further processing of the slurry such as subsequent incorporation of the slurry into the consumer product. It may also negatively influence the aspect of the consumer product.

Agglomeration is defined as a process of contact and adhesion whereby dispersed microcapsules are held together by weak physical interactions. Most of the agglomerates in a microcapsule slurry contain clusters of two or three microcapsules. The process may ultimately lead to phase separation by creaming of the microcapsules at the surface of the slurry or the formation of precipitates of larger than colloidal size. Agglomeration is a reversible process.

In order to obtain a homogenous consumer product, which shows substantially no sign of flocculation, coagulation, creaming or sedimentation it is customary in the industry to dilute the slurry with water and to filter the diluted microcapsule slurry through sieves of appropriate size before incorporating it into the consumer product. If agglomeration of the microcapsules has taken place in the slurry, for example during storage, filtration of the diluted slurry through a sieve of a size that is about two or three times the size of the microcapsules may result in compaction of the microcapsule on the sieve and ultimately blockage of the sieve, leading to lower amount of functional material being present in the consumer product.

This problem has been generally solved by employing sieves of sizes that are substantially larger than about two or three times the size of the microcapsules. However, not all producers are equipped with sieves of sizes which could accommodate microcapsule agglomerates of relatively large sizes. Moreover, even if the sieve blockage may be avoided by using larger size sieves, the consumer product might show undesirable signs of flocculation, coagulation, creaming or sedimentation.

Therefore, a problem exists to prevent agglomeration of microcapsules in a microcapsule slurry, thereby circumventing the problem of sieve blockage and preventing an undesirable aspect of the consumer product.

The applicant has surprisingly and unexpectedly found that adding a monovalent and/or divalent inorganic salt to the slurry of microcapsules comprising a plurality of core-shell microcapsules comprising a core comprising at least one fragrance ingredient and a shell encapsulating the core, wherein the core-shell microcapsules represent about 25 wt % to about 50 wt % of the composition; and an aqueous phase, prevents the agglomeration of the microcapsules upon dilution with water.

### SUMMARY OF THE INVENTION

In a first aspect, the present invention provides a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt % of the composition; and an aqueous phase, wherein the aqueous phase comprises a monovalent and/or a divalent inorganic salt, wherein the conductivity of the microcapsule composition is above about 5000 μS/cm.

In a further aspect, the invention provides a method of making a microcapsule composition as described herein.

A method of preventing the flocculation of microcapsules as defined herein, wherein a monovalent and/or a divalent inorganic salt is added to a microcapsule composition in the form of a slurry is also provided in a further aspect.

In another aspect, the use of a monovalent and/or a divalent inorganic salt to prevent flocculation in a microcapsule composition as described herein is provided.

A further aspect is concerned with a consumer product comprising a microcapsules composition as defined herein.

### Definitions

The term “functional material” refers to any substance which, when added to a product, may improve the perception of this product by a consumer or may enhance the action of this product in an application. Examples of functional materials include perfume or fragrance ingredients, bioactive agents (such as bactericides, insect repellents and pheromones), substrate enhancers (such as silicones and brighteners), enzymes (such as lipases and proteases), dyes and pigments, and combinations thereof.

The microcapsule size is generally defined by its median particle size by volume also known as volume median diameter (Dv50), which represents the maximum particle diameter below which 50% of the sample volume exists.

The conductivity of a solution is a measure of the ability to conduct electricity. Conductivity measurements are used routinely in many industrial and environmental applications as a reliable way of measuring the ionic content in a solution. In many cases, conductivity is linked directly to the total dissolved solids (or the concentration of ions) in that solution. Ionic compounds, when dissolved in water, dissociate into ions. High quality deionized water has a conductivity of about 0.05 μS/cm at 25° C., typical drinking water is in the range of 200-1000 μS/cm, while sea water is about 50 mS/cm (or 50,000 μS/cm). The total electrolyte concentration in solution affects the behaviour of the microcapsules suspended or dispersed in solution.

A bio-based polymer useful in the formation of microcapsule compositions according to the present invention can be any polymer that is obtained or derived from a natural source, such as plant, fungus, bacterium, algae or animal sources that may be native, i.e. unmodified from their natural state, or chemically modified, and which is capable of forming an encapsulating shell around a functional material.

All percentages are expressed as weight percentages (wt.-%) unless otherwise indicated.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

The applicant has surprisingly and unexpectedly found that adding a monovalent and/or a divalent inorganic salt to a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt % of the composition; and an aqueous phase, wherein the conductivity of the microcapsule composition is above about 5000 μS/cm, results in a microcapsule composition in the form of a slurry which does not show any signs of microcapsule agglomeration and passes through a sieve of a size of about two to three times the volume average diameter (Dv50) of the microcapsules without blocking the sieve.

The invention, therefore, provides a microcapsule composition in the form of a slurry, comprising a plurality of core-shell microcapsules comprising a core comprising at least one functional material and a shell encapsulating the core, wherein the core-shell microcapsules represent about 25 wt % to about 50 wt %, preferably about 30 wt % to about 40 wt % of the composition; and an aqueous phase, wherein the aqueous phase comprises a monovalent and/or a divalent inorganic salt, wherein the conductivity of the microcapsule composition is above about 5000 μS/cm.

### Core-Shell Microcapsules

The microcapsules of the present invention are core-shell microcapsules comprising a core comprising a functional material and a shell encapsulating the core.

Core-shell microcapsule compositions are generally provided in the form of a slurry, that is, a dispersion or suspension of microcapsules in an aqueous medium, that may contain in the order of above 50 wt.-% of water, preferably above about 60 wt.-% of water.

The slurry may be used as such (i.e. neat, in an undiluted form) or diluted, typically in deionized or tap water. The diluted slurries may contain in the order of 50 to 99 wt.-% water, depending on the dilution factor.

In one embodiment, the shell of the core-shell microcapsules comprises a polymer selected from the group consisting of a melamine-formaldehyde polymer, a urea-formaldehyde polymer, a polyurea, a polyurethane, a polyamide, a polyacrylate, a polycarbonate, and mixtures thereof, as defined hereinabove.

### Thermosetting Resins

Thermosetting resins are typically obtained by reacting polyfunctional monomers, such as amines, isocyanates, alcohols or phenols, chlorocarboxylic acids, (meth)acrylates, epoxides, silanes and aldehydes.

Thermosetting resins, such as aminoplast, polyurea and polyurethane resins, as well as combinations thereof are commonly employed as shell materials in the preparation of core-shell microcapsules. They are particularly valued for their resistance to leakage of the benefit agent when dispersed in aqueous suspending media, even in surfactant-containing media.

In one embodiment, the shell may comprise a melamine-formaldehyde polymer. This type of core-shell capsule has proved to be particularly suitable for benefit agent encapsulation and is described, for instance in WO 2018/197266 A1, WO 2016/207180 A1, and WO 2017/001672 A1.

In one embodiment, the shell may comprise a polyurea or polyurethane polymer. Also this type of core-shell capsule has been successfully used for benefit agent encapsulation and has the advantage to address consumer concerns with regard to residual formaldehyde in the composition. Such capsules are also described, for instance in WO 2016/071149 A1.

In one embodiment, the shell may comprise, a polyacrylate, one or more monoethylenically unsaturated and/or polyethylenically unsaturated monomer(s) in polymerized form. This type of core-shell capsule has also been successfully used for benefit agent encapsulation. Such capsules are described in the prior art, for instance in WO 2013/111912 A1 or WO 2014/032920 A1.

### Polymeric Stabilizer

In one embodiment, the shell may comprise a polymeric stabilizer that is formed by combination of a polymeric surfactant with at least one aminosilane, such as the shells described in WO 2020/233887A1.

### Coacervates

In one embodiment, the shell can comprise a complex coacervate formed of at least one protein and at least one polysaccharide. Such core-shell capsules have proved suitable for functional material encapsulation and are described, for instance in WO 1996/020612 A1, WO 2001/03825 A1 or WO 2015/150370 A1.

Cross-linking of at least one protein with a first cross-linking agent followed by the addition of at least one polysaccharide to form a complex coacervate is described in WO 2021/239742 A1.

### Hydrated Polymer Phase and Polymeric Stabilizer

In one embodiment, the shell may comprise a hydrated polymer phase and a polymeric stabilizer at an interface between the shell and the core, as described in patent application WO 2023/020883A1.

In such an arrangement, the polymeric stabilizer provides an impervious encapsulating material, whereas the bio-based hydrated polymer phase provides the desired deposition and adherence to the substrate.

The polymeric stabilizer may be selected from a broad range of film-forming materials and resins. Preferably, the polymeric stabilizer is highly cross-linked, in order to decrease significantly the diffusion of the encapsulated functional material through the shell. Preferably the imperviousness of the shell is sufficiently high to significantly prevent the leakage of the functional material in extractive base, such as consumer products comprising surfactants.

In one embodiment of the present invention, the polymeric stabilizer is a thermosetting resin.

Thermosetting resins are typically obtained by reacting polyfunctional monomers, such as amines, isocyanates, alcohols or phenols, chlorocarboxylic acids, (meth)acrylates, epoxides, silanes and aldehydes.

In one embodiment of the present invention, the polymeric stabilizer is formed by reaction of an aminosilane with a polyfunctional isocyanate. Such a polymeric stabilizer has the advantage of being highly crosslinked and susceptible of providing surface anchoring groups that can be used to immobilize additional materials to complete shell formation. These additional materials may comprise additional encapsulating materials, coatings and, as described in more details hereinafter, simple and complex coacervate, and hydrogels.

The aminosilane employed in the formation of the polymeric stabilizer can be selected from a compound of Formula (I).

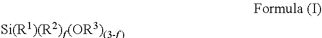

wherein R¹ is a linear or branched alkyl or alkenyl residue comprising an amine functional group; R² is each independently a linear or branched alkyl group with 1 to 4 carbon atoms; R³ is each independently a H or a linear or branched alkyl group with 1 to 4 carbon atoms; and f is 0, 1 or 2.

The silane groups may undergo polycondensation reactions with one another to form a silica network at the oil/water interface that additionally stabilizes this interface.

In one embodiment, R² and R³ are each independently methyl or ethyl.

In one embodiment, f is 0 or 1.

In one embodiment, R¹ is a C₁-C₁₂ linear or branched alkyl or alkenyl residue comprising an amine functional group. Optionally, R¹ is a C₁-C₄ linear or branched alkyl or alkenyl residue comprising an amine functional group.

In one embodiment, the amine functional group is a primary, a secondary or a tertiary amine.

In one embodiment, the at least one aminosilane is a bipodal aminosilane. By “bipodal aminosilane” it is meant a molecule comprising at least one amino group and two residues, each of these residues bearing at least one alkoxysilane moiety. Bipodal aminosilanes are particularly advantageous for forming stable oil-water interfaces, compared to conventional aminosilanes. Without wishing to be bound by theory, it is believed that this beneficial role is due to the particular, bi-directional arrangement of the silane moieties in the molecule of a bipodal aminosilane, which allows formation of a more tightly linked silica network at the oil-water interface.

In one embodiment, the bipodal aminosilane is a compound of Formula (II).

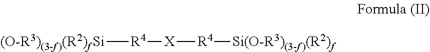

wherein X is —NR⁵—, —NR⁵—CH₂—NR⁵—, —NR⁵—CH₂—CH₂—NR⁵—, —NR⁵—CO—NR⁵—, or

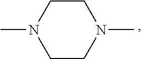

R² is each independently a linear or branched alkyl group with 1 to 4 carbon atoms;
R³ is each independently H or a linear or branched alkyl group with 1 to 4 carbon atoms;
R⁴ is each independently a linear or branched alkylene group with 1 to 6 carbon atoms;
R⁵ is each independently H, CH₃ or C₂H₅; and
f is each independently 0, 1 or 2.

In one embodiment, R² is CH₃ or C₂H₅.

In one embodiment, R³ is CH₃ or C₂H₅.

In one embodiment, R⁴ is —CH₂—, —CH₂—CH₂— or —CH₂—CH₂—CH₂—.

In one embodiment, R⁵ is H or CH₃.

In one embodiment, f is 0 or 1.

Examples of suitable bipodal aminosilanes include, but are not limited to, bis(3-(triethoxysilyl)propyl)amine, N,N′-bis(3-(trimethoxysilyl)propyl)urea, bis(3-(methyldiethoxysilyl) propyl)amine, N,N′-bis(3-(trimethoxysilyl)propyl)ethane-1,2-diamine, bis(3-(methyldimethoxysilyl)propyl)-N-methylamine, N,N′-bis(3-(triethoxysilyl) propyl)piperazine, and combinations thereof.

In one embodiment, the bipodal aminosilane is bis(3-(triethoxysilyl)propyl)amine, which has the advantage of releasing ethanol instead of more toxic and less desirable methanol during the polycondensation of the ethoxysilane groups.

The bipodal aminosilane can be a secondary aminosilane. Using a secondary bipodal aminosilane instead of a primary aminosilane decreases the reactivity of the polymeric stabilizer with respect to electrophilic species, in particular aldehydes. Hence, functional materials containing high levels of aldehydes may be encapsulated with a lower propensity for adverse interactions between core-forming and shell-forming materials.

Other aminosilanes may also be used in combination with the aforementioned bipodal aminosilanes, in particular the aminosilanes described hereinabove.

The polyfunctional isocyanate may be selected from organic isocyanates, in which an isocyanate group is bonded to an organic residue (R—N═C═O or R—NCO). The polyfunctional isocyanate may be selected from alkyl, alicyclic, aromatic and alkylaromatic, as well as anionically modified polyfunctional isocyanates, with two or more (e.g. 3, 4, 5, etc.) isocyanate groups in a molecule, and mixtures thereof.

Preferably, the polyfunctional isocyanate is an aromatic or an alkylaromatic isocyanate, the alkylaromatic polyfunctional isocyanate having preferably methylisocyanate groups attached to an aromatic ring. Both aromatic and methylisocyanate-substituted aromatic polyfunctional isocyanates have a superior reactivity compared to alkyl and alicyclic polyfunctional isocyanates. Among these, 2-ethylpropane-1,2,3-triyl tris((3-(isocyanatomethyl)phenyl)carbamate) is particularly preferred, because of its trifunctional nature that favors the formation of intermolecular cross-links and because of its intermediate reactivity that favors network homogeneity. This alkylaromatic polyfunctional isocyanate is commercially available under the trademark Takenate D-100 N, sold by Mitsui or under the trademark Desmodur® Quix175, sold by Covestro.

As an alternative to aromatic or alkylaromatic polyfunctional isocyanates, it may also be advantageous to add an anionically modified polyfunctional isocyanates, because of the ability of such polyfunctional isocyanates to react at the oil/water interface and even in the water phase close to the oil/water interface. A particularly suitable anionically modified polyfunctional isocyanate has Formula (III).

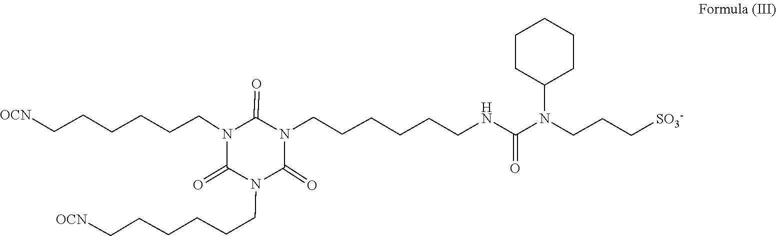

Formula (III) shows a commercially available anionically modified polyisocyanate, which is a modified isocyanurate of hexamethylene diisocyanate, sold by Covestro under the trademark Bayhydur® XP2547.

In a preferred embodiment of the present invention, polyfunctional isocyanate is 2-ethylpropane-1,2,3-triyl tris((3-(isocyanatomethyl)phenyl)carbamate). Particularly preferably, the polymeric stabilizer is formed by reaction of bis(3-(triethoxysilyl)propyl)amine and 2-ethylpropane-1,2,3-triyl tris((3-(isocyanatomethyl)phenyl)carbamate). The combination of this particular bipodal secondary aminosilane and polyfunctional isocyanate provides advantageous interface stability and release properties. The stabilized interface is sufficiently impervious to effectively encapsulate the at least one functional material comprised in the core and possesses the desired surface functional groups.

The hydrated polymer phase can be a coacervate, in particular a complex coacervate.

By “complex coacervation” is meant the formation of an interfacial layer comprising a mixture of polyelectrolytes.

The phenomenon of coacervation may be observed under a light microscope, wherein it is marked by the appearance of a ring around the core composition droplet. This ring consists of the aforementioned polyelectrolyte-rich phase that has a different refractive index than the surrounding aqueous phase.

The coacervation of a polyelectrolyte is generally induced by bringing the polyelectrolyte to its isoelectric point, meaning the point where the net charge of the polyelectrolyte is zero or close to zero. This may be achieved by changing the salt concentration or the pH of the medium. In a complex coacervation, complexation occurs at the pH where one of the polyelectrolytes has an overall positive electrical charge (polycation), whereas the other polyelectrolyte has an overall negative charge (polyanion), so that the overall electrical charge of the complex is neutral.

In preferred embodiments of the present invention, the coacervate may be formed from a polycation and a polyanion.

Preferably, the pH is used as parameter driving the coacervation. Thus, the polycation preferably has a pH-dependent electrical charge. This is the case for polymers bearing primary, secondary and tertiary amino groups, such as polyamines, for example chitosan, and most proteins, for example gelatin. Proteins have the additional advantage of being prone to temperature-dependent structural transitions that may also be used to control the morphology of the coacervates. In particular, varying the temperature of some proteins may induce the formation of secondary, tertiary or quaternary structures of the protein that may also be used to control the properties of the coacervate.

Chitosan has the advantage of being derived from chitin, which is a natural polymer.

In preferred embodiments of the present invention, the polycation is selected from the group consisting of proteins, chitosan, and combinations thereof.

More particularly, the polycation can be a protein selected from the group consisting of gelatin, casein, albumin, polylysine, soy proteins, pea proteins, rice proteins, hemp proteins, and combinations thereof.

In particularly preferred embodiments of the present invention, the at least one protein is a gelatin, even more preferably a Type B gelatin.

Type B gelatin can be obtained from the alkaline treatment of collagen and is well known for its ability to form complexes with anionic polyelectrolytes, such as negatively charged polysaccharides under mild acidic conditions.

Gelatin is usually characterized by the so-called “Bloom Strength”. In the context of the present invention, the Bloom Strength refers to the rigidity of a gelatin film, as measured by so-called “Bloom Gelometer”, according to the Official Procedures of the Gelatin Manufacturers Institute of America, Inc., revised 2019, Chapter 2.1. According to this procedure, the Bloom Strength, expressed in Bloom, is equal to the weight, expressed in g, required to move vertically a standardized plunger, having a diameter of 12.5 mm, to a depth of 4 mm into a gelatin gel, which has been prepared under controlled conditions, i.e. by dissolving 6.67 wt.-% of gelatin in deionized water at 60° C., in a standardized jar, and letting the gel form for 17 hours at 10° C. The higher the weight is, the higher is the Bloom Strength of the gelatin used for making the tested gel.

In preferred embodiments of the present invention, the Type B gelatin has a Bloom Strength of 90 to 250 Bloom.

If the Bloom Strength is too low, the gel is mechanically weak and coacervates obtained therefrom may not form a self-standing layer of gelatin-rich phase around the core composition. If the Bloom Strength is too high, then the coacervates and the gelatin-rich phase obtained therefrom may be too brittle.

In preferred embodiments of the present invention, the Type B gelatin is obtainable from fish, because fish gelatin meets better acceptance within consumer than beef or pork gelatin, mainly due to health concerns, sociological context or religious rules.

Alternatively, the protein may be a vegetable protein, in particular a pea protein and/or a soy protein, which have the advantage of being vegan.

The polycation may be a denaturated protein. In the contrary to native proteins, denaturated proteins have been deprived from their ability to form secondary, tertiary or quaternary structures and are essentially amorphous. Such amorphous proteins may form more impervious films compared to native proteins and therefore also contribute to the encapsulating power of the shell. Denaturation may be achieved by treating the protein with chemical or physical means, such as acid or alkaline treatment, heat or exposure to hydrogen bond disrupting agents.

In cases where the polycation is chitosan, the chitosan can have a molecular weight between 3,000 and 1,000,000 g/mol, more particularly between 10,000 and 500,000 g/mol, still more particularly between 30,000 and 300,000 g/mol.

The polyanion may be any negatively charged polymer. However, as the pH is preferably used to control coacervation, it may be more advantageous that the electrical charge of the polymer is pH-dependent. Such polymer may be selected from polymers having pendent carboxylic groups, such as methacrylic acid and acrylic acid polymers and copolymers, hydrolyzed maleic anhydride copolymers and polysaccharides bearing carboxylic groups.

In preferred embodiments of the present invention, the polyanion is a polysaccharide comprising carboxylate groups and/or sulfate groups.

Polysaccharides comprising carboxylate groups are particularly suitable for complex coacervation with proteins. This is due to the fact that the net electrical charge of these polysaccharides may be adjusted by adjusting the pH, so that the complexation with ampholytic proteins is facilitated. Complexation occurs at the pH where the protein has an overall positive electrical charge, whereas the polysaccharide as an overall negative charge, so that the overall electrical charge of the complex is neutral. These polysaccharides include native polysaccharides, i.e. unmodified from nature, and modified polysaccharides.

The polysaccharide comprising carboxylic acid groups may comprise uronic acid units, in particular hexuronic acid units. Such polysaccharides are broadly available in nature.

The hexuronic acid units can be selected from the group consisting of galacturonic acid units, glucuronic acid units, in particular 4-O-methyl-glucuronic acid units, guluronic acid units, mannuronic acid units, and combinations thereof.

The polysaccharide comprising carboxylic acid groups may be branched. Branched polysaccharides comprising carboxylic acid groups have the advantage of forming more compact networks than linear polysaccharides and therefore may favor the imperviousness of the encapsulating shell, resulting in reduced leakage and greater encapsulation efficiency.

The carboxylate groups can be at least partially present in the form of the corresponding carboxylate salt, in particular the corresponding sodium, potassium, magnesium or calcium carboxylate salt.

In particular embodiments of the present invention, the polyanion is selected from the group consisting of pectin, gum arabic, alginate, and combinations thereof.

Among the pectins, the carboxylic acid groups can be partially present in the form of the corresponding methyl ester. The percentage of carboxylic acid groups that are present in the form of the corresponding methyl ester can be from 3% to 95%, preferably from 4% to 75%, more preferably from 5 to 50%. Pectins comprising carboxylic groups, of which 50% or more are present in the form of the corresponding methyl ester, are referred to as “high methoxylated”. Pectins comprising carboxylic acid groups, of which less than 50% are present in the form of the corresponding methyl ester, are referred to as “low methoxylated”.

Among the two variants of gum Arabic, i.e. gum acacia Senegal and gum acacia Seyal, gum acacia Senegal is preferred, owing to the higher level of glucuronic acid in gum acacia Senegal.

The hydrated polymer phase can be a hydrogel.

In the context of the present invention, a “hydrogel” is a three-dimensional (3D) network of hydrophilic polymers that can swell in water, while maintaining the structure due to chemical or physical cross-linking of individual polymer chains.

A hydrogel can be formed by several methods at interfaces, especially by self-assembly of polyelectrolytes around existing interfaces, covalent grafting of hydrogel particles in solution, polymerization of hydrosoluble monomers initiated at the interface and phase separation of water soluble macromolecules onto the interface.

To avoid any ambiguity, in context of the present invention, a coacervate, especially a complex coacervate, which is cross-liked, in particular by covalent bonds, is considered as a hydrogel.

The applicant has found that the use of hydrogels particularly enhances both the deposition and adherence of microcapsules on substrates, in particular on fabrics.

The hydrogel can be interlinked with the polymeric stabilizer, in particular via the functional groups present on the surface of this stabilizer.

This allows the locking of the hydrogel layer onto the polymeric stabilizer present at droplet interface, making the shell composed of a polymer composite, instead of only a blend.

Both hydrogel cross-linking and hydrogel interlinking with the polymeric stabilizer may be performed sequentially or simultaneously.

In preferred embodiments of the present invention, the hydrogel is a crosslinked coacervate, in particular a complex coacervate crosslinked with polyfunctional aldehyde, more particularly a difunctional aldehyde selected from the group consisting of succinaldehyde, glutaraldehyde, glyoxal, benzene-1,2-dialdehyde, benzene-1,3-dialdehyde, benzene-1,4-dialdehyde, piperazine-N,N-dialdehyde, 2,2′-bipyridyl-5,5′-dialdehyde, and combinations thereof. Difunctional aldehydes are known to be effective cross-linking agents for proteins.

The hydrogel can be thermosensitive and possess a gelation temperature, in particular between 20° C. and 50° C., preferably between 25° C. and 40° C. When using such a hydrogel, the deposition performance of the capsules on fabric can increase, when washing the fabric at a temperature which is above hydrogel gelation temperature.

The shell can be further stabilized with a stabilizing agent. Preferably the stabilizing agent comprises at least two carboxylic acid groups. Even more preferably, the stabilizing agent is selected from the group consisting of citric acid, benzene-1,3,5-tricarboxylic acid, benzene-1,2,4-tricarboxylic acid, 2,5-furandicarboxylic acid, itaconic acid, poly(itaconic acid) and combinations thereof.

### Polymeric Stabilizer, Hydrocolloid and Linker

In one embodiment, the shell may comprise a polymeric stabilizer formed by combination of a polymeric surfactant with at least one aminosilane; a hydrocolloid and a linker derived from a epoxy resin as described in application GB2203193,4.

In one embodiment, the polymeric surfactant comprises a polysaccharide comprising carboxylic acid groups. The polysaccharide comprising carboxylic acid groups are as defined hereinabove.

The at least one aminosilane is defined as hereinabove.

In one embodiment, the polymeric stabilizer further comprises a polyfunctional isocyanate. The polyfunctional isocyanate is as defined hereinabove.

### Hydrocolloid and Epoxy Resin

Hydrocolloids contain a large number of hydroxyl groups, leading to their high affinity for water molecules. They have been used as wall material in microencapsulation processes both in the food industry and beyond. Most naturally sourced hydrocolloids comprise a number of polysaccharides and certain proteins (e.g. gelatin).

The hydrocolloid may interact with the polymeric stabilizer by physical forces, physical interactions, such as hydrogen bonding, ionic interactions, hydrophobic interactions or electron transfer interactions.

The presence of a linker derived from an epoxy resin leads to reinforcing of the shell by covalent or physical bonding, with the hydrocolloid and/or with the polymeric surfactant. Therefore, a combination of epoxy resin with a hydrocolloid in the microcapsule formulation helps to stabilize the oil/water interface.

In one embodiment, the hydrocolloids are plant or animal-derived hydrocolloids or gelatin from animal-derived collagen.

Suitable plant-derived hydrocolloids may be pectin, modified starches, guar gum, locust bean gum, and konjac mannan, along with exudate gums, such as gum arabic, gum ghatti, and tragacanth, and seaweed-derived hydrocolloids, such as agar, alginates, and carrageenan.

In one embodiment, the hydrocolloid is selected from the group consisting of pectin, modified starch and gelatin.

Examples of suitable epoxy resin from which the linker is derived include but are not limited to epoxidized unsaturated oils such as epoxidized soybean oil, epoxidized vegetable oil, and the like; epoxidized alcohols such as isoborbide glycidyl ether, ethylene glycol diglycidyl ether, diethylene glycol diglycidyl ether, polyethylene glycol diglycidyl ether, polypropylene glycol diglycidyl ether, glycerol polyglycidyl ether, diglycerol polyglycidyl ether, polyglycerol polyglycidyl ether, polyglycerol-3-glycidyl ether, trimethylolpropane polyglycidyl ether, neopentyl glycol diglycidyl ether, 1,6-hexanediol diglycidyl ether, pentaerythritol polyglycidyl ether; castor oil glycidyl ether; epoxidized polysaccharides such as sorbitol polyglycidyl ether; epoxidised phenols such as resorcinol diglycidyl ether, hydrogenated bisphenol A diglycidyl ether; diglycidyl terephthalate; diglycidyl o-phthalate; N-glycidyl phthalimide; epoxy cresol novolac resin; hexahydrophthalic acid diglycidyl ester; epoxidised terpenes and the like.

In one embodiment, the shell comprises one essentially homogenous layer.

In one embodiment, the shell comprises two or more discrete layers.

In one embodiment, the shell comprises two or more gradual and non-discrete layers.

In one embodiment, the shell of the microcapsules can be made of a biodegradable material or a non-biodegradable material. In one embodiment, the microcapsules are made of a biodegradable material.

In one embodiment, the volume median diameter Dv(50) of the plurality of core-shell microcapsules is from 1 to 100 μm, preferably 2 to 75 μm, more preferably 5 to 60 μm, even more preferably 20 to 50 μm. Microcapsules having volume median diameter in the range from 10 to 30 μm show optimal deposition on various substrates, such as fabrics and hair. The volume median diameter is measured by static light scattering involving laser diffraction particle size analysis.

Encapsulated compositions are presented in the form of a slurry of microcapsules suspended in an aqueous suspending medium, comprising between about 25 wt % to about 50 wt %, preferably about 30 wt % to about 40 wt % of core-shell material.

The resulting encapsulated composition, presented in the form of a slurry of microcapsules suspended in an aqueous suspending medium, may be incorporated in a consumer product base as such or it may be diluted with water prior to incorporation in a consumer product.

Fragrance ingredients A comprehensive list of fragrance ingredients that may be encapsulated in accordance with the present invention may be found in the perfumery literature, for example “Perfume & Flavor Chemicals”, S. Arctander (Allured Publishing, 1994). Encapsulated fragrance ingredients according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy)butan-2-olAMBERKETAL (3,8,8,11a-tetramethyldodecahydro-1H-3,5a-epoxynaphtho[2,1-c]oxepine); AMBERMAX (1,3,4,5,6,7-hexahydro-.beta.,1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1 S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl)propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl)quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4(5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl)hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl) acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA (1-(2,6,6-trimethyl-1-cyclohex-3-enyl)but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5(6H)-ylidene)butanal); EBANOL ((E)-3-methyl-5-(2,2,3-trimethylcyclopent-3-en-1-yl)pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl)ethanone); FLORALOZONE (3-(4-ethylphenyl)-2,2-dimethylpropanal); FLORHYDRAL (3-(3-isopropylphenyl)butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl)pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl)phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl)methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy)hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl)propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl)propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl)butanamide); PEACH PURE (5-heptyldihydrofuran-2(3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl)acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl)propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2(3H)-one); RADJANOL SUPER ((E)-2-ethyl-4-(2,2,3-trimethylcyclopent-3-en-1-yl)but-2-en-1-ol); RASPBERRY KETONE (4-(4-hydroxyphenyl)butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl)pentan-2-ol); SCENTAURUS CLEAN (ethyl (Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl)ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl)pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl)propan-2-ol); TERPINEOL ALPHA (2-(4-methyl-1-cyclohex-3-enyl)propan-2-ol); TERPINEOL PURE (2-(4-methylcyclohex-3-en-1-yl)propan-2-ol); TERPINOLENE (1-methyl-4-(propan-2-ylidene)cyclohex-1-ene); TERPINYL ACETATE (2-(4-methyl-1-cyclohex-3-enyl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

The at least one fragrance ingredient may comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

### Functional Material

Suitable functional materials to be incorporated into the core of the core-shell microcapsules in addition to the fragrance ingredients include flavor ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

The at least one functional material may comprise at least one functional cosmetic ingredient. The functional cosmetic ingredients for use in the encapsulated composition are preferably hydrophobic. Preferably, the cosmetic ingredients have a calculated octanol/water partition coefficient (ClogP) of 1.5 or more, more preferably 3 or more. Alternatively preferred, the ClogP of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Particularly useful functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C18-Sphingosine, CAS-No: 100403-19-8).

The at least one functional material may comprise agents which suppress or reduce malodour and its perception by adsorbing odour, agents which provide a warming or cooling effect, insect repellents or UV absorbers.

In a microcapsule composition according to the present invention, the proportion of the functional material can be between about 10 to about 99 wt.-%, preferably between about 50 to about 95 wt.-%, even more preferably between about 70 to about 90 wt.-%, relative to the total weight of the solid content of the microcapsule composition.

In the context of the present invention, the solid content is measured by using a thermo-balance operating at 120° C. The solid content, expressed as weight percentage of the initial microcapsule composition deposited on the balance was taken at the point where the drying-induced rate of weight change had dropped below 0.1%/min.

Monovalent and/or Divalent Inorganic Salt

It has been surprisingly and unexpectedly found that adding a monovalent and/or a divalent inorganic salt to a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt %, preferably about 30 wt % to about 40 wt % of the composition; and an aqueous phase, wherein the conductivity of the resulting microcapsule composition in the form of a slurry is above about 5000 μS/cm, results in a microcapsule composition in the form of a slurry which does not show any signs of microcapsule agglomeration and passes through a sieve of a size of about two to three times the volume average diameter (Dv50) of the microcapsules upon dilution with water, without blocking the sieve.

In one embodiment, the monovalent and/or divalent inorganic salt is selected from the group consisting of a lithium salt, a sodium salt, a potassium salt, a calcium salt, a magnesium salt, an ammonium salt and a mixture thereof.

In one embodiment, the monovalent and/or divalent inorganic salt is a chloride, a sulfate, a carbonate, a bicarbonate, or a mixture thereof, preferably wherein the monovalent and/or divalent salt is a chloride.

In one embodiment, the monovalent and/or divalent inorganic salt is calcium chloride or magnesium chloride, preferably calcium chloride.

In one embodiment, the monovalent and/or divalent inorganic salt is completely dissolved in the aqueous phase.

In one embodiment, the concentration of the monovalent and/or divalent inorganic salt is between 0.01 wt.-% to 2.0 wt.-% with respect to the neat microcapsule slurry. Optionally, the concentration of the monovalent and/or divalent inorganic salt is between 0.1 wt.-% to 1.5 wt.-% with respect to the neat microcapsule slurry. Optionally, the concentration of the monovalent and/or divalent inorganic salt is between 0.15 wt.-% to 1.0 wt.-%, optionally between 0.2 wt.-% to 0.8 wt.-% with respect to the microcapsule slurry.

In one embodiment, the concentration of the monovalent and/or divalent inorganic salt depends on the nature of the monovalent and/or divalent inorganic salt. In one embodiment, the concentration of calcium chloride is above about 0.3% wt.-% with respect to the microcapsule slurry.

In one embodiment, the concentration of the monovalent and/or divalent inorganic salt depends on the nature of the shell of the microcapsules.

In one embodiment, the microcapsule composition in the form of a slurry comprises a plurality of core-shell microcapsules comprising a core comprising at least one fragrance ingredient, wherein the shell comprises a hydrated polymer and a polymeric stabilizer formed by reaction of an aminosilane with a polyfunctional isocyanate, wherein the hydrated polymer is a coacervate, in particular a complex coacervate, optionally a complex coacervate formed from a polycation and a polyanion, wherein the core-shell microcapsules represent about 28 wt % to about 34 wt %, preferably about 32 wt % of the composition; and an aqueous phase, wherein the aqueous phase comprises a monovalent and/or a divalent inorganic salt in a concentration of between 0.1 wt.-% to 1.0 wt.-% with respect to the microcapsule composition, wherein the aqueous phase represents about 62 wt % to about 68 wt % of the total weight of the slurry.

In one embodiment, the inorganic salt is calcium chloride. In one embodiment, the concentration of calcium chloride is above about 0.3% wt.-% with respect to the microcapsule slurry.

The conductivity of the resulting microcapsule composition in the form of a slurry is above about 5000 μS/cm.

In one embodiment, water is deionized water or tap water.

In one embodiment, the microcapsule composition may further comprise water, optionally deionized water, wherein the volumetric ratio microcapsule composition to water is of 1 to above about 0.5.

In one embodiment, the volumetric ratio microcapsule composition to water, optionally deionized water is of 1 to about 0.5.

In one embodiment, the volumetric ratio microcapsule composition to water, optionally deionized water is of 1 to about 1.

In one embodiment, the volumetric ratio microcapsule composition to water, optionally deionized water is of 1 to about 2.

In one embodiment, the volumetric ratio microcapsule composition to water, optionally deionized water is of 1 to about 4.

In one embodiment, the volumetric ratio microcapsule composition to water, optionally deionized water is of 1 to about 6.

In one embodiment, the volumetric ratio microcapsule composition to water, optionally deionized water is below about 10.

In one embodiment, the conductivity of the microcapsule composition further comprising water, optionally deionized water is above about 2100 μS/cm.

### Methods

The invention further provides a method of making a microcapsule composition as defined hereinabove, comprising the steps of
a) providing a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt %, preferably about 30 wt % to about 40 wt % of the composition;
b) optionally, adding water to the microcapsule composition of step a), wherein the volumetric ratio microcapsule composition of step a) to water is of 1 to above about 0.5;
c) adding a monovalent and/or a divalent inorganic salt to the microcapsule composition of step a) or step b); and
d) optionally adding water to the microcapsule composition resulting from step c), wherein the volumetric ratio microcapsule composition resulting from step c) to water is of 1 to above about 0.5;
wherein when step b) is not carried out, the conductivity of the microcapsule composition resulting after step c) is above about 5000 μS/cm;
optionally wherein the conductivity of the microcapsule composition resulting from step d) is above about 2100 μS/cm.

The core-shell microcapsules and the monovalent and/or a divalent inorganic salt are as defined hereinabove.

Optionally, the water is deionized water.

The monovalent and/or a divalent inorganic salt is added to the slurry provided in step a) in an amount such that the resulting conductivity of the microcapsule composition is above about 5000 μS/cm.

In one embodiment, the monovalent and/or a divalent inorganic salt is added to the microcapsule composition provided in step a) in an amount such that the resulting concentration of the monovalent and/or divalent inorganic salt is between 0.01 wt.-% to 2.0 wt.-% with respect to the undiluted, neat slurry of microcapsules provided in step a). Optionally, the concentration of the monovalent and/or divalent inorganic salt is between 0.1 wt.-% to 1.5 wt.-% with respect to the slurry of microcapsules provided in step a). Optionally, the concentration of the monovalent and/or divalent inorganic salt is between 0.15 wt.-% to 1.0 wt.-%, optionally between 0.2 wt.-% to 0.8 wt.-%.

In one embodiment, the microcapsule composition is optionally further diluted by adding water, wherein the volumetric ratio microcapsule composition to water is of 1 to above about 0.5. In one embodiment, the conductivity of the microcapsule composition further comprising water is above about 2100 μS/cm.

In one embodiment, the microcapsule composition provided in step a) is first diluted by adding water, wherein the volumetric ratio of microcapsule composition to water is of 1 to above about 0.5, before adding the monovalent and/or the divalent inorganic salt. In one embodiment, the conductivity of the microcapsule composition comprising water is above about 2100 μS/cm.

A method of preventing the flocculation of a microcapsule composition as defined herein is provided, wherein a monovalent and/or a divalent inorganic salt is added to a microcapsule composition in the form of a slurry comprising a plurality of core-shell microcapsules comprising a core comprising at least one fragrance ingredient and a shell encapsulating the core, wherein the core-shell microcapsules represent about 25 wt % to about 50 wt %, preferably about 30 wt % to about 40 wt % of the composition, and an aqueous phase; and wherein the conductivity of the microcapsule composition is above about 5000 μS/cm.

In one embodiment, the salt is added as an aqueous solution to a microcapsule composition in the form of a slurry comprising a plurality of core-shell microcapsules comprising a core comprising at least one functional material and a shell encapsulating the core.

In one embodiment, the salt is added as a solid to a microcapsule composition in the form of a slurry comprising a plurality of core-shell microcapsules comprising a core comprising at least one functional material and a shell encapsulating the core.

In yet another aspect, it is provided the use of a monovalent and/or a divalent inorganic salt to prevent flocculation in a microcapsule composition as defined herein.

### Consumer Product

Another aspect of the present invention provides a consumer product comprising a microcapsule composition as described herein.

The consumer product may be selected from the group consisting of household (home) care, personal care, fabric care and pet care products.

Suitable home care products include hard surface cleaners, heavy duty detergents and detergent powders, air care compositions.

Suitable personal care products include cleansing compositions (such as shampoos, bath and shower gels, liquid soaps, soap bars), conditioning compositions (such as hair care conditioners), bath and shower lotions, oral care compositions, deodorant compositions, antiperspirant compositions, skin care products

Suitable fabric care compositions include laundry care detergents, laundry care conditioners, fabric refreshers, scent boosters.

Encapsulated compositions according to the present invention are particularly useful when employed as perfume delivery vehicles in consumer goods that require, for delivering optimal perfumery benefits, that the microcapsules adhere well to a substrate on which they are applied. Such consumer goods include hair shampoos and conditioners, as well as textile-treatment products, such as laundry detergents and conditioners.

The encapsulated composition of the present invention, presented in the form of a slurry of microcapsules suspended in an aqueous suspending medium may be incorporated as such in a consumer product base.

The present invention is further illustrated by means of the following non-limiting examples:

### EXAMPLES

The volume median diameter Dv(50) of the microcapsules was measured by static light scattering involving laser diffraction particle size analysis using a Malvern Mastersizer 2000S Particle Size Analyzer.

Conductivity was measured using a SevenMulti pH Conductivity Meter at 25° C.

### Example 1.1: Microcapsule Slurry Preparation (S1)

A microcapsule slurry was prepared according to a modified method disclosed in WO 2023/020883A1, Example 1, which is shown below:
a) A core composition was prepared by admixing 0.7 g of bipodal aminosilane (bis(3-triethoxysilylpropyl)amine), 50 g Takenate D-110N (ex Mitsui) and 39 g of fragrance composition;
b) The core composition obtained in step a) was emulsified in a mixture of 1.0 g high methoxylated grade pectin (of type APA 104, ex Roeper) in 73 g of water by using a 300 ml reactor and a cross-beam stirrer with pitched beam operating at a stirring speed of 600 rpm at a temperature of 25° C. for 10 min;
c) The temperature of the system was raised to 85° C. over 4 hours, 0.3 g of trimesic acid (1,3,5-benzenetricarboxylic acid) were added and the system was maintained at this temperature for 1.5 h while maintaining stirring as in step b);
d) The system was slowly cooled to 40° C., while maintaining stirring as in step b); e) At a temperature of 40° C., 10 g of a 10% gelatin solution in water was added, while maintaining stirring as in step b);
f) The system was slowly cooled to 10° C., while maintaining stirring as in step b);
g) The slurry of core-shell capsules obtained in step f) was finally let to stabilize at room temperature.

The solid content of the slurry obtained was 33 wt.-%, the volume median size (d50) of the capsules was 32 μm and the encapsulation efficiency of 99%.

### Example 1.2: Microcapsule Slurry Preparation (S2)

A microcapsule slurry was prepared according to a method disclosed in WO 2021/213930A1. The shell of these capsules comprises a resin formed by reaction of at least one trifunctional araliphatic isocyanate with at least one partially alkylated amino-aldehyde pre-condensate.

The solid content of the slurry obtained was 43 wt.-%, the volume average size (D50) of the capsules was 10 μm and the encapsulation efficiency was 100%.

### Example 1.3: Microcapsule Slurry Preparation (S3)

A microcapsule slurry was prepared according to a method disclosed in WO 2017/001672 A1. The shell of these capsules comprises a network of cross-linked aminoplast resin, wherein 75-100 wt % of the resin comprises 50-90 wt % of a terpolymer and from 10-50 wt % of a polymeric stabilizer; the terpolymer comprising:
(a) from 20-35 wt % of moieties derived from at least one triamine,
(b) from 30-60 wt % of moieties derived from at least one diamine,
(c) from 20-35 wt % of moieties derived from the group consisting of alkylene and alkylenoxy moieties having 1 to 6 methylene units.

The volume median size (d50) of the capsules was 25 μm.

### Example 2: Conductivity Measurements, Dilution and Filtration Results

### 2.1. Neat Slurries

Conductivity measurements of slurries S1, S2 and S3 are shown in Table 1.

TABLE 1

| entry | slurry | Conductivity (μS/cm) |
|---|---|---|
| 1 | Slurry of Example 1.1 (S1) | 2600 |
| 2* | Slurry of Example 1.2 (S2) | 7500 |
| 3 | Slurry of Example 1.3 (S3) | 4800 |

*comparative

2.2. Dilution with Water in a 1:2 Ratio (Volumetric)

Slurries S1, S2 and S3 were diluted with deionized water (DI) in a ratio (volume) slurry S:DI water=1:2 and the conductivities and the behavior of the dilute slurries when passed through a sieve of 75 μm is shown in Table 2 (entries 3-S1, 5-S2 and 6-S3). For completeness, the conductivity of tap water and DI water were also recorded (entries 1 and 2, respectively). It can be observed that diluted slurry S1, with conductivity 900 μS/cm (entry 3), blocked the sieve, whereas the diluted slurry S2, with conductivity 3800 μS/cm (entry 5), passed though the sieve without leaving any residue on the sieve. Like S1, diluted slurry S3, with conductivity of 1500 μS/cm (entry 6), blocked the sieve.

Therefore, a relationship exists between the conductivity of a neat slurry and the behaviour of the slurry when diluted with water with respect to a sieve that is two to three times the size of the microcapsules.

Since tap water is known to have higher conductivity than DI water due to the presence of traces of salts, the neat slurry S1 was subsequently diluted with tap water in a ratio (volume) S1:tap water=1:2. The conductivity and the behavior of the tap water diluted slurry when passed through a sieve of 75 μm is shown in Table 2 (entry 4). As expected, the conductivity of the slurry S1 diluted with tap water is higher than the conductivity of the slurry S1 diluted with DI water (1300 μS/cm vs 900 μS/cm), however the tap water diluted slurry also did not pass completely through the 75 μm sieve without leaving any residue on the sieve.

When 100 g of S1 diluted with deionized water (entry 3) was filtered through a sieve of 75 μm, 85 g of dry microcapsules were recovered from the sieve. However, the amount of residue observed on the sieve after passing the tap water diluted slurry S1 was smaller than the amount of residue observed after passing the DI water diluted slurry. Subsequently, to the slurry S1 diluted with deionized water as above (S1:DI=1:2, entry 3), various monovalent and/or divalent inorganic salts were added as a solid (entries 7 to 10) and the conductivities of the diluted slurries were measured. The diluted slurries were passed through a sieve of 75 μm and their behaviour recorded.

TABLE 2

| | | Added | | |
|---|---|---|---|---|
| | | monovalent | Conduc- | 75 μm |
| | | and/or divalent | tivity | sieve |
| entry | Solution/slurry | inorganic salt ^{a} | (μS/cm) | pass |
| 1 | Deionized water (DI) | n/a | 8 | n/a |
| 2 | Tap water | n/a | 520 | n/a |
| 3 | S1:DI = 1:2 | n/a | 900 | no |
| 4 | S1:tap water = 1:2 | n/a | 1300 | no |
| 5* | S2:DI = 1:2 | n/a | 3800 | yes |
| 6 | S3:DI = 1:2 | n/a | 1500 | no |
| 7 | S1:DI = 1:2, +NaCl | NaCl, 1% | 12000 | yes |
| 8 | S1:DI = 1:2, +Na₂SO₄ | Na₂SO₄, 1% | 3600 | yes |
| 9 | S1:DI = 1:2, +MgCl₂ | MgCl₂, 0.5% | 4430 | yes |
| 10 | S1:DI = 1:2, +CaCl₂ | CaCl₂, 0.5% | 5600 | yes |

^{a} the salt concentration (%, wt) refers to the concentration of the salt in the slurry S1 before dilution with water;
*comparative

Dilution of the slurry S1 with deionized water in a ratio slurry:DI water=1:2 and subsequent addition of different inorganic salts (entries 7 to 10) resulted in significantly increased conductivity of the diluted slurry with the result that the diluted slurries passed through the 75 μm sieve leaving no residue on the sieve.

For example, 100 g of slurry S1 was diluted with 200 g deionized water. 0.5 g of MgCl₂ was added to the diluted slurry under stirring. The stirring was carried out over 10 minutes (entry 9). The diluted slurry was filtered through a sieve of 75 μm. Less than 5 g of residue was recovered from the sieve.

In another set of experiments, solid calcium chloride was added in various amounts into slurries S1 and S3, under stirring (Table 3). The conductivities of the resulting slurries were measured. Subsequently, each of the slurries was diluted with DI water, in a ratio (volume) slurry:DI water=1:2. The diluted slurries were passed through a sieve of 75 μm and their behaviour on the sieve was recorded.

TABLE 3

| | | | | |
|---|---|---|---|---|
| | | concentration | | |
| | | of the salt in | | 75 μm |
| | | the slurry S1 | Conductivity | sieve |
| entry | Solution/slurry | (wt %) | (μS/cm) | pass |

S1

| | | | | |
|---|---|---|---|---|
| 1 | S1 + CaCl₂ | 0.1 | 2900 | n/a |
| | (S1 + CaCl₂):DI = 1:2 | | 1600 | no |
| 2 | S1 + CaCl₂ | 0.2 | 4600 | n/a |
| | (S1 + CaCl₂):DI = 1:2 | | 2000 | no |
| 3 | S1 + CaCl₂ | 0.3 | 5800 | n/a |
| | (S1 + CaCl₂):DI = 1:2 | | 2600 | yes |
| 4 | S1 + CaCl₂ | 0.5 | 8700 | n/a |
| | (S1 + CaCl₂):DI = 1:2 | | 4300 | yes |

S3

| | | | | |
|---|---|---|---|---|
| 5 | S3 + CaCl₂ | 0.5 | 9100 | n/a |
| | (S3 + CaCl₂):DI = 1:2 | | 3900 | yes |

To the slurry as prepared in Example 1.1 (S1) was added solid calcium chloride in different amounts (entries 1 to 4, top lines) under stirring until the salt was completely dissolved. The conductivities of the slurries increased compared to the neat slurry S1 (2600 μS/cm, Table 1, entry 1).

As expected, subsequent dilution of the slurries containing salt with DI water (entries 1 to 4, bottom lines) led to increased conductivities of the diluted slurries compared to the conductivity of the slurry S1 diluted with DI water only in 2:1 ratio (900 μS/cm, Table 2, entry 3).

Likewise, to slurry S3 it was also added 0.5 wt % solid calcium chloride (entry 5, top line), to the effect that the conductivity of the slurry increased from 4800 μS/cm (neat S3, Table 1, entry 3) to 9100 μS/cm when 0.5% CaCl₂ is present in the slurry. Dilution of this slurry with DI water (1:2) resulted in a dilute slurry with significantly higher conductivity (3900 μS/cm) than the 1:2 diluted slurry S3 (1500 μS/cm, Table 2, entry 6).

From Table 3 it can be observed that subsequent dilution of the slurry containing added salt with DI water in a ratio salted slurry:DI water=1:2 led to conductivity-dependent behavior of the diluted slurries when passed through a75 μm sieve. Conductivity of a dilute slurry of below about 2000 μS/cm is not sufficient for the slurry to pass through a 75 μm sieve without blocking it.

2.3. Dilution with Water in Different Ratios

Slurry S1 to which 0.5 wt % CaCl₂ was added, with conductivity of 8700 μS/cm (Table 3, entry 4, top line, shown again in Table 4, entry 1) was diluted with DI water in different ratios. Likewise, slurry S3 to which 0.5 wt % CaCl₂ was added, with conductivity of 9100 μS/cm (Table 3, entry 5, top line, shown again in Table 4, entry 6) was diluted with DI water in different ratios.

The conductivities of the resulting diluted slurries were measured and the diluted slurries were passed through a sieve of 75 μm, their behaviour on the sieve being recorded (Table 4).

TABLE 4

| | | | |
|---|---|---|---|
| | | | 75 μm |
| | | Conductivity | sieve |
| entry | Dilution | (μS/cm) | pass |

S1

| | | | |
|---|---|---|---|
| 1 | S1 + 0.5% CaCl₂ | 8700 | n/a |
| 2 | (S1 + 0.5% CaCl₂):DI = 1:0.5 | 5400 | yes |
| 3 | (S1 + 0.5% CaCl₂):DI = 1:1 | 5000 | yes |
| 4 | (S1 + 0.5% CaCl₂):DI = 1:2 | 4300 | yes |
| 5 | (S1 + 0.5% CaCl₂):DI = 1:4 | 2800 | yes |

S3

| | | | |
|---|---|---|---|
| 6 | S3 + 0.5% CaCl₂ | 9100 | n/a |
| 7 | (S3 + 0.5% CaCl₂):DI = 1:2 | 3900 | yes |
| 8 | (S3 + 0.5% CaCl₂):DI = 1:4 | 2800 | yes |
| 9 | (S3 + 0.5% CaCl₂):DI = 1:6 | 1900 | no |

These results confirm that a conductivity of above at least about 2100 μS/cm is required for a diluted slurry containing added salt and diluted with water in a ratio of 1:more than about 0.5 in order to pass through a 75 μm sieve without blocking it.

It can also be concluded that the conductivity of a neat, undiluted slurry containing added salt of at least about 5000 μS/cm is required so that when diluted with water, in a ratio of 1:more than about 0.5, the diluted slurry passes through a sieve of 75 μm sieve without blocking it.

1. A microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt % of the composition; and an aqueous phase, wherein the aqueous phase comprises a monovalent and/or a divalent inorganic salt, and wherein the conductivity of the microcapsule composition is above about 5000 μS/cm.

2. A c composition comprising a microcapsule composition according to claim 1, further comprising water, wherein the volumetric ratio microcapsule composition according to claim 1 to water is of 1 to above about 0.5.

3. The composition according to claim 1, wherein the shell of the microcapsule comprises a melamine-formaldehyde polymer; an urea-formaldehyde polymer; a polyurea or polyurethane polymer; a polyamide; a polyacrylate; a polycarbonate; a polymeric stabilizer that is formed by combination of a polymeric surfactant with at least one aminosilane; a complex coacervate formed by cross-linking of at least one protein with a first cross-linking agent and at least one polysaccharide; or a hydrated polymer and a polymeric stabilizer formed by reaction of an aminosilane with a polyfunctional isocyanate; or a polymeric stabilizer formed by combination of a polymeric surfactant with at least one aminosilane, a hydrocollolid and a linker derived from an epoxy resin.

4. The composition according to claim 1, wherein the shell of the microcapsule comprises
a) a complex coacervate formed by cross-linking of at least one protein with a first cross-linking agent and at least one polysaccharide; or
b) hydrated polymer and a polymeric stabilizer formed by reaction of an aminosilane with a polyfunctional isocyanate; or
c) a polymeric stabilizer formed by combination of a polymeric surfactant with at least one aminosilane, a hydrocollolid and a linker derived from an epoxy resin.

5. The composition according to claim 1, wherein the shell of the microcapsule comprises a hydrated polymer and a polymeric stabilizer formed by reaction of an aminosilane with a polyfunctional isocyanate, wherein the hydrated polymer is a coacervate.

6. The composition according to claim 1, wherein the volume average diameter (Dv50) of the microcapsules is between about 5 micron to about 60 micron.

7. The composition according to claim 1, wherein the core further comprises a functional material selected from the group consisting of flavour ingredients, cosmetic ingredients, bioactive agents, substrate enhancers, enzymes, dyes and pigments, nutraceuticals and combinations thereof.

8. The composition according to any claim 1, wherein the monovalent and/or divalent inorganic salt is selected from the group consisting of a lithium salt, a sodium salt, a potassium salt, a calcium salt, a magnesium salt, an ammonium salt and a mixture thereof.

9. The composition according to claim 1, wherein the monovalent and/or divalent inorganic salt is a chloride, a sulfate, a carbonate, a bicarbonate, or a mixture thereof.

10. The composition according to claim 1, wherein the concentration of the monovalent and/or divalent inorganic salt is between 0.01 wt.-% to 2.0 wt.-% with respect to the microcapsule composition.

11. A method of making a composition according to claim 1, comprising the steps of:
a) providing a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt % of the composition;
c) adding a monovalent and/or a divalent inorganic salt to the microcapsule composition of step a)
wherein conductivity of the microcapsule composition resulting after step c) is above about 5000 μS/cm.

12. A method of preventing the flocculation of a microcapsule composition according to claim 1, wherein a monovalent and/or a divalent inorganic salt is added to a microcapsule composition in the form of a slurry comprising a plurality of core-shell microcapsules comprising a core comprising at least one fragrance ingredient and a shell encapsulating the core, wherein the core-shell microcapsules represent about 25 wt % to about 50 wt % and an aqueous phase; and wherein the conductivity of the microcapsule composition is above about 5000 μS/cm.

13. A method of preventing flocculation in a microcapsule composition according to claim 1, the method comprising the incorporation of amonovalent and/or divalent organic salts into the microcapsule composition.

14. A consumer product comprising a microcapsules composition as defined in claim 1.

15. A method of preventing flocculation in a microcapsule composition according to claim 2, the method comprising the incorporation of amonovalent and/or divalent organic salts into the microcapsule composition.

16. The composition according to claim 2, wherein the conductivity of the composition is above about 2100 μS/cm.

17. A method of making a composition according to claim 2, comprising the steps of:
a) providing a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt % of the composition;
c) adding a monovalent and/or a divalent inorganic salt to the microcapsule composition of step a) or step b); and
d) adding deionized water to the microcapsule composition resulting from step c), wherein the volumetric ratio microcapsule composition resulting from step c) to deionized water is of 1 to above about 0.5;
wherein the conductivity of the microcapsule composition resulting after step c) is above about 5000 μS/cm.

18. A method of making a composition according to claim 2, comprising the steps of:
a) providing a microcapsule composition in the form of a slurry, comprising a plurality of core-shell microcapsules comprising a core comprising at least one fragrance ingredient and a shell encapsulating the core, wherein the core-shell microcapsules represent about 25 wt % to about 50 wt % of the composition;
b) adding deionized water to the microcapsule composition of step a), wherein the volumetric ratio microcapsule composition of step a) to deionized water is of 1 to above about 0.5;
c) adding a monovalent and/or a divalent inorganic salt to the microcapsule composition of step b).
